(19) United States
(12) Patent Application Publication
(10) Pub. No.: US 2025/0177072 A1
(43) Pub. Date: Jun. 5, 2025

(54) APPARATUS FOR POSITIONING A TOOL

(71) Applicant: THE QUEEN'S UNIVERSITY OF BELFAST, Belfast (GB)

(72) Inventors: Yan JIN, Belfast (GB); Yinglun JIAN, Belfast (GB); Mark PRICE, Belfast (GB); Dan SUN, Belfast (GB); Aislinn MCALLEENAN, Newry (GB)

(21) Appl. No.: 18/845,043

(22) PCT Filed: Mar. 9, 2023
(86) PCT No.: PCT/GB2023/050561

§ 371 Date: Sep. 9, 2024

(30) Foreign Application Priority Data
Mar. 9, 2022 (GB) 2203245.2

(51) Int. Cl. A61B 34/00

(52) U.S. Cl. CPC A61B 34/71

(57) ABSTRACT

An apparatus for positioning a tool, the apparatus comprising a base, a platform for supporting the tool, and three spherical arms each connecting the platform to the base. Each spherical arm comprises a first spherical linkage and a second spherical linkage. Each first spherical linkage is connected to the base by a primary joint, and all primary joints are coaxial with one another. Each first spherical linkage is connected to the respective second spherical linkage by a first revolute joint. Each second spherical linkage is connected to the platform by a second revolute joint. Axes of the primary joints, first revolute joints and second revolute joints intersect at a remote centre of motion that is remote from the platform. The apparatus further comprises rotary inputs arranged on the base to receive rotary drive to rotate each first spherical linkage about the respective primary joint so as to move the platform over a portion of the surface of a notional sphere and rotate the platform about a tool axis that is normal to the surface of the platform. Particularly, the platform can have infinite rolling motion about the tool axis in any achievable orientation.

This invention relates to an apparatus for positioning a tool. In particular, but not exclusively, the tool may be a surgical tool and the apparatus may be used to manipulate the tool during a surgical procedure.

### BACKGROUND

In minimally invasive surgical (MIS) procedures, long and thin surgical tools are typically used to complete all the surgical steps. MIS procedures involve the use of small incisions at the surgical site, consequently resulting in only small scars, little blood loss and rapid patient recovery. These advantages make MIS a popular procedure that is widely applied to many surgeries including but not limited to laparoscopic surgery, heart surgery, endovascular surgery, cancer surgery, and eye surgery.

However, in order to conduct manual MIS successfully, surgeons have to face problems including limited vision, unexpected hand tremor, existing leverage effect, lack of force feedback, restricted degree of freedom, and the risk of tearing or otherwise damaging surrounding skin or soft tissues. The assistance of specially designed robots can effectively improve precision, ensure safety, and avoid hand tremor, bringing capabilities beyond those of the skilled surgeon to help mitigate these limitations. The motion during MIS procedures typically requires four degree of freedom motion including three rotations (3R) about the incision point and one translation (1T) through the incision point. The conventional way to achieve this kind of motion is to apply a two degree of freedom remote centre-of-motion (RCM) mechanism with an additional two actuators mounted on its end-effector (i.e. the platform supporting the surgical tool). The two degree of freedom RCM mechanism can orient its end-effector in a two rotational degree of freedom motion about a fixed virtual point, the “remote centre of motion” (RCM) point. The additional two actuators mounted on the end-effector provide one translational degree of freedom motion and one rotational degree of freedom motion, i.e. the rolling motion.

A disadvantage of such a configuration, however, is that the total inertia of the system is high due to the presence of the two actuators mounted on the end-effector. Also, the precision of the system is affected by the high inertia of the structure. In addition, the actuators mounted on the end-effector increase the bulkiness and complexity of the system due to additional transmission and cables for control and power supply.

An example of such an apparatus is described in US 2004/0024387 A1 (Payandeh et al.). Payandeh et al. describes a method and apparatus for a spherical parallel mechanism. The apparatus has a platform and at least three kinetic chains. The kinetic chains each comprise a link with one end pivotally coupled to the platform about a first axis and a second end pivotally coupled to an arm about a second axis. The arms in all of the kinetic chains share a common third axis. All of the first, second and third axes for all of the kinetic chains pass through a stationary point in space. Movement of the arms to selected angular positions about the common third axis adjusts an orientation and position of the platform about a spherical surface centered at the stationary point. In order to provide rotational movement of an implement fitted to the platform, a rotational actuator is provided on the platform. The arrangement of Payandeh et al, thus provides two degrees-of-freedom by virtue of the rotational implement actuator and a linear actuator mounted on the platform, and a further two degrees-of-freedom by virtue of the kinematic chains.

It is an object of certain embodiments of the present invention to overcome or mitigate certain disadvantages associated with the prior art.

### BRIEF SUMMARY OF THE DISCLOSURE

In accordance with an aspect of the present invention there is provided an apparatus for positioning a tool, the apparatus comprising:
a base;
a platform for supporting the tool;
three spherical arms each connecting the platform to the base, each spherical arm comprising a first spherical linkage and a second spherical linkage, wherein:
   (i) each first spherical linkage is connected to the base by a primary joint, and all primary joints are coaxial with one another;
   (ii) each first spherical linkage is connected to the respective second spherical linkage by a first revolute joint;
   (iii) each second spherical linkage is connected to the platform by a second revolute joint; and
   (iv) axes of the primary joints, first revolute joints and second revolute joints intersect at a remote centre of motion that is remote from the platform; and
rotary inputs arranged on the base to receive rotary drive to rotate each first spherical linkage about the respective primary joint so as to move the platform over a portion of the surface of a notional sphere and rotate the platform about a tool axis that is normal to the surface of the platform.

In certain embodiments, the three spherical arms may be configured such that all extreme permitted positions of the tool axis relative to the axes of the primary joints are outside of a conical workspace that is centred on and symmetrical about the axes of the primary joints.

For four extreme positions, 1, 2, 3, 4 of the tool axis, a cone angle of the workspace may be equal to twice the minimum angle amongst θ₁, θ₂, θ₃, and θ₄, wherein:

$$\{ \begin{matrix}{θ}_{1} = - α + β + γ \\ {θ}_{2} = α - β + γ \\ {θ}_{3} = α + β - γ \\ {θ}_{4} = α + β + γ\end{matrix} ;$$

and
α is the angle between the primary joints and a respective first revolute joint, β is the angle between the first revolute joint and a respective second revolute joint, and γ is the angle between the second revolute joint and the tool axis.

The three spherical arms may be configured such that α=β=γ and θ₁=θ₂=θ₃.

In certain embodiments, the apparatus may additionally comprise a tool supported by the platform. In certain embodiments, the tool may be a surgical tool.

In certain embodiments, the apparatus may further comprise one or more cables extending from the base to the platform, wherein the one or more cables may each be driven to move the tool supported on the platform to provide a further degree of freedom of the tool. The one or more cables may extend from the base to the platform over one or more pulleys mounted to none, one or more than one of the three spherical arms.

The further degree of freedom of the tool may comprise one or more of translation of the tool along the tool axis, a grabbing motion of the tool or a clamping motion of the tool. In other embodiments, the further degree of freedom of the tool may comprise other movements of the tool.

In certain embodiments, the further degree of freedom may comprise translation of the tool along the tool axis, and the apparatus may further comprise a leadscrew and a nut threaded on the leadscrew, wherein the nut may support the tool relative to the platform and may be translatable along the leadscrew so as to move the tool along the tool axis in response to the leadscrew being driven by one of the one or more cables.

In certain embodiments, one or more of the three spherical arms may comprise recesses to accommodate the one or more cables.

In certain embodiments, one or more of the primary joints, the first revolute joints and the second revolute joints may be hollow so as to permit the passage of the one or more cables.

The apparatus may comprise one or more auxiliary actuators arranged on the base and each may be configured to drive the one of the one or more cables.

The apparatus may comprise one or more primary actuators arranged on the base and each may be configured to provide rotary drive to one of the rotary inputs.

In certain embodiments, the platform may have infinite rolling motion about the tool axis in any achievable orientation.

### BRIEF DESCRIPTION OF THE DRAWINGS

Embodiments of the invention are further described hereinafter with reference to the accompanying drawings, in which:

FIG. 1 is a perspective view of an apparatus for positioning a tool in accordance with an embodiment of the present invention;

FIG. 2 is a side view of the apparatus of FIG. 1 and illustrates the workspace of the apparatus;

FIG. 3 is a perspective view corresponding to the view of FIG. 2;

FIG. 4 is a side view of a part of the apparatus of FIG. 1;

FIG. 5 illustrates the four extreme positions of the apparatus of FIG. 1;

FIG. 6 shows part of an apparatus according to an embodiment of the present invention that is configured such that the workspace is maximised;

FIG. 7 is a perspective view of an apparatus according to an alternative embodiment of the present invention;

FIG. 8 is a perspective view of an apparatus according to another embodiment of the present invention;

FIG. 9 is a perspective view of an apparatus according to another embodiment of the present invention;

FIG. 10 is a perspective view of an apparatus according to another embodiment of the present invention; and

FIG. 11 is a perspective view of an apparatus according to another embodiment of the present invention.

### DETAILED DESCRIPTION

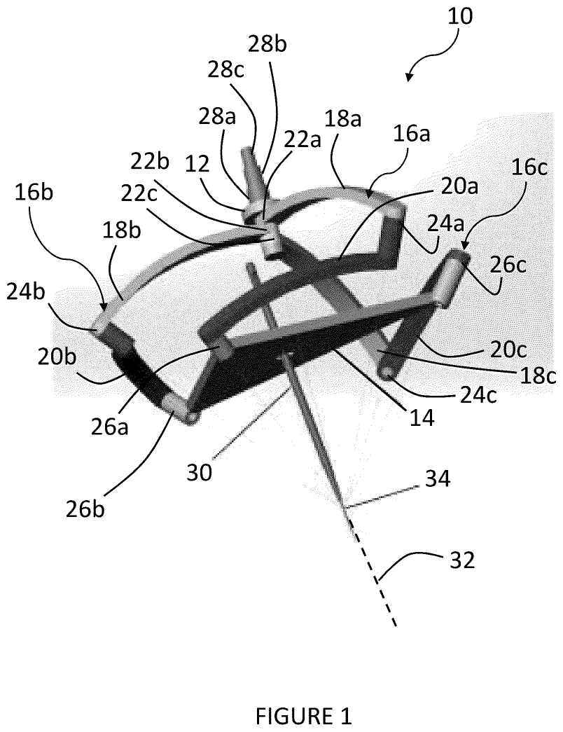

FIG. 1 shows an apparatus 10 according to an embodiment of the present invention The apparatus 10 is a parallel mechanism and may be used to position a tool 30. In certain embodiments, the tool 30 may be a surgical tool and the apparatus 10 may be used to manipulate the tool 30 as part of a surgical procedure. As is explained below, certain embodiments of the present invention are particularly suited for use in minimally invasive surgical procedures. Other embodiments may be utilized for other surgical and non-surgical applications wherein a tool requires positioning and manipulation.

The apparatus 10 comprises a base 12, a platform 14 for supporting the tool 30, and three spherical arms 16a, 16b, 16c connecting the platform 14 to the base 12. In embodiments, the apparatus 10 comprises precisely three spherical arms 16a, 16b, 16c. Each of the spherical arms 16a, 16b, 16c comprises a first spherical linkage 18a, 18b, 18c and a second spherical linkage 20a, 20b, 20c connected to one another by a first revolute joint 24a, 24b, 24c.

Each of the first spherical linkages 18a, 18b, 18c is connected to the base 12 by a primary joint 22a, 22b, 22c. All of the primary joints 22a, 22b, 22c are coaxial with one another. The primary joints 22a, 22b, 22c are also revolute joints.

Each of the second spherical linkages 20a, 20b, 20c is connected to the platform by a second revolute joint 26a, 26b, 26c. All of the primary joints 22a, 22b, 22c, first revolute joints 24a, 24b, 24c and second revolute joints 26a, 26b, 26c permit 360° relative rotation about their respective axes.

The axes (i.e. of rotation) of the primary joints 22a, 22b, 22c, first revolute joints 24a, 24b, 24c and second revolute joints 26a, 26b, 26c all intersect with one another at a so-called remote centre of motion 34. The remote centre of motion 34 is remote from the platform 14 such that it is separated by a distance from the platform 14 and does not coincide with any part of the platform 14. The remote centre of motion 34 coincides with the tool 30 supported centrally by the platform 14 when the tool 30 extends along a tool axis 32 that extends perpendicularly from the surface of the platform 14.

Indeed, the term “spherical linkage” refers to an arm that extends between and is connected to two revolute joints, whose axes intersect at a point that is the remote centre of motion 34. When two or more spherical linkages are connected together, the axes of all the revolute joints intersect at this common point, i.e. the remote centre of motion 34. The form of the spherical linkages are not limited to any particular shape and include but are not limited to arcuate shapes and polyline shapes. In certain embodiments, it is preferable for the spherical linkages to have a form that facilitates easy manufacture and/or avoids collision with other spherical linkages that are connected thereto when moving relative to one another. In the non-limiting embodiment shown in FIGS. 1 to 7 and 10 to 11, each spherical linkage follows an arc of a notional circle (or, indeed, a path on the surface of a notional sphere). In certain embodiments of the present invention, each of the first spherical linkages 18a, 18b, 18c and the second spherical linkages 20a, 20b, 20c may have a different radius of curvature relative to the others for each spherical arm 16a, 16b, 16c. This may ensure that each of the first spherical linkages 18a, 18b, 18c and the second spherical linkages 20a, 20b, 20c may avoid collision with the others. In certain embodiments, the radii of curvature of the first spherical linkages 18a, 18b, 18c and the second spherical linkages 20a, 20b, 20c may be sorted from largest to smallest with reference to the respective spherical linkage as: 18a, 18b, 18c, 20c, 20b, 20a.

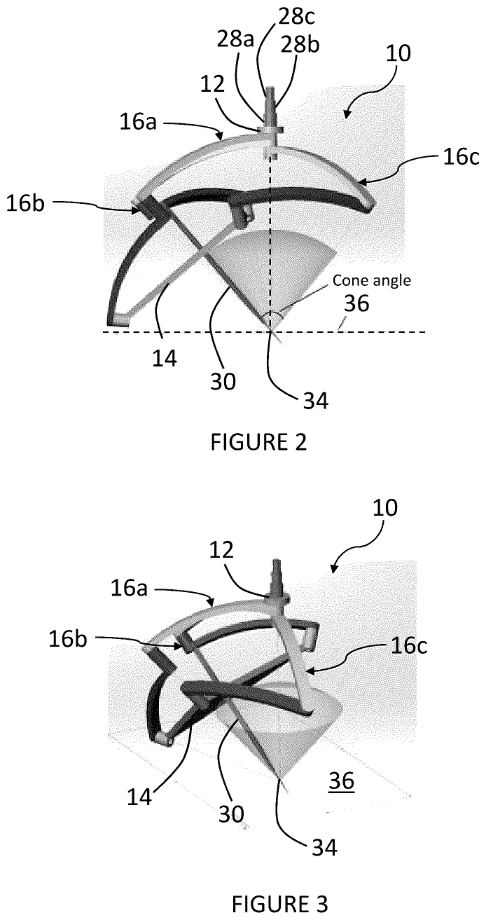

Rotary inputs 28a, 28b, 28c are provided on the base 12 and are arranged to receive rotary drive (e.g. from any suitable actuator) for moving the spherical arms 16a, 16b, 16c and, hence, the platform 14 and any tool 30 supported thereon. In particular, rotary input 28a may receive rotary drive to rotate the first spherical linkage 18a about the axis of the primary joint 22a. Similarly rotary input 28b may receive rotary drive to rotate the first spherical linkage 18b about the axis of the primary joint 22b, and rotary input 28c may receive rotary drive to rotate the first spherical linkage 18c about the axis of the primary joint 22c. By rotating the three first spherical linkages 18a, 18b, 18c about the primary joints 22a, 22b, 22c the second spherical linkages 20a, 20b, 20c are caused to move relative to the first spherical linkages 18a, 18b, 18c by rotating about the first revolute joints 24a, 24b, 24c. Consequently, the platform 14 is caused to move, and such movement includes tangential movement along a portion of the surface of a notional sphere that is centred on the remote centre of motion 34 due to the interactions of the spherical arms 16a, 16b, 16c, their anchoring to both the base 12 and the platform 14, the presence and orientation of the first revolute joints 24a, 24b, 24c and the presence and orientation of the second revolute joints 26a, 26b, 26c. The portion of the notional sphere whose surface the platform 14 may move along is termed the workspace of the apparatus 10. As illustrated in the side view of FIG. 2, the workspace has a cone angle centred on the axes of the primary joints 22a, 22b, 22c such that the workspace is conical and symmetrical about such axes (which are coaxial with one another). A plane 36 is defined perpendicularly relative to the axes of the primary joints 22a, 22b, 22c, where such plane 36 contains the remote centre of motion 34. FIG. 3 shows a perspective view of the apparatus 10 in the configuration of FIG. 2.

The axes of rotation of the primary joints 22a, 22b, 22c are coaxial with one another and aligned with radii of the notional circles along which the first spherical linkages 18a, 18b, 18c extend. Similarly, the axes of rotation of the first revolute joints 24a, 24b, 24c are aligned with (different) radii of the notional circles along which the first spherical linkages 18a, 18b, 18c extend, and radii of the notional circles along which the second spherical linkages 20a, 20b, 20c extend. The axes of rotation of the second revolute joints 26a, 26b, 26c are aligned with (different) radii of the notional circles along which the second spherical linkages 20a, 20b, 20c extend.

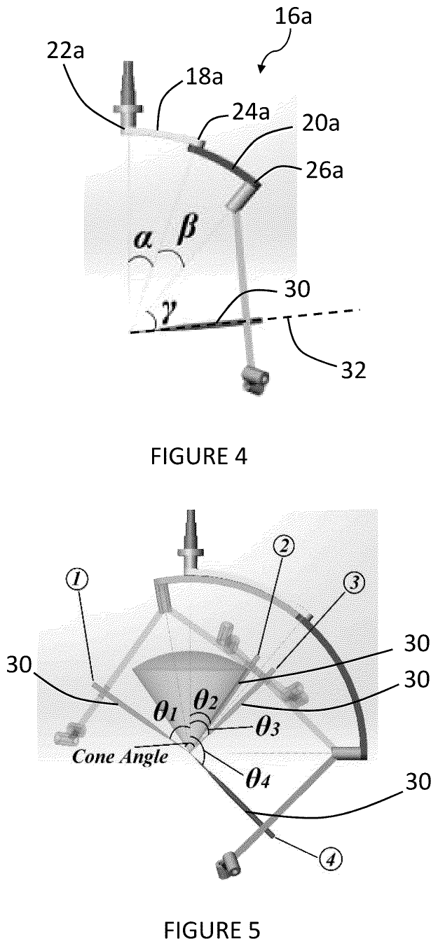

As shown in FIG. 4 with reference to the spherical arm 16a, the length and radius of curvature of the first spherical linkage 18a defines a first angle α between the axes of the primary joint 22a and the first revolute joint 24a. The length and radius of curvature of the second spherical linkage 20a defines a second angle β between the axes of the first revolute joint 24a and the second revolute joint 26a. A third angle γ is defined between the axis of the second revolute joint 26a and the tool axis 32. Equivalent angles are defined for the spherical arms 16b and 16c.

A fourth angle, θ, may be defined between the axis of the primary joint 22a and the tool axis 32. Given that the first spherical linkage 18a, the second spherical linkage 20a, and the platform 14 (and hence the tool axis 32) are each moveable relative to one another due to the primary joint 22a, the first revolute joint 24a and the second revolute joint 26a, four extreme positions 1, 2, 3, 4 of the tool 30 and the tool axis 32 relative to axis of the primary joint 22a are defined according to the relative arrangement of first, second and third angles, α, β and γ. These four extreme positions 1, 2, 3, 4 are shown superimposed on one another in FIG. 5 and are defined with reference to the fourth angle, θ, below.

$$\{ \begin{matrix}{θ}_{1} = - α + β + γ \\ {θ}_{2} = α - β + γ \\ {θ}_{3} = α + β - γ \\ {θ}_{4} = α + β + γ\end{matrix}$$

“Singularity” exists when any two of the first spherical linkages 18a, 18b, 18c and the corresponding second spherical linkages 20a, 20b, 20c are coplanar with one another. It is preferable for singularities to be avoided because one or more degree of freedom will be gained or lost at a singular configuration so that the motion cannot be accurately controlled. In order for the workspace to be singularity free, the four extreme positions 1, 2, 3, 4 of the tool 30 should be outside of the workspace.

In addition to the platform 14 (and hence tool 30) being moveable with two degrees of freedom about the surface of the workspace, the above described apparatus 10 permits rotation of the platform 14 (and tool 30) about the tool axis 32, thus providing three degrees of freedom. In particular, by providing the centre axis of the workspace in alignment with the axes of the primary joints 22a, 22b, 22c and the coaxial rotary inputs 28a, 28b, 28c, infinite rolling motion about the tool axis 32 in either direction (i.e. both clockwise and anticlockwise) is possible at any position within the workspace. That is to say, the tool 30 may be infinitely rotated in either direction by the apparatus 10 when within the workspace.

Notably, the three degrees of freedom is provided for without the need for any actuators on the platform 14 or tool 30, or any of the other part moving relative to the base 12. Indeed, as noted above, any and all actuators may be provided on the base 12 in order to drive the rotary inputs 28a, 28b, 28c. Consequently, embodiments of the present invention advantageously permit small inertia, a small footprint, high stiffness, and a compact design. By reducing total inertia, the precision of control with respect to manipulation of the tool 30 is improved.

The above-mentioned cone angle of the workspace is defined as twice the minimum angle amongst θ₁, θ₂, θ₃, and θ₄. It is necessarily the case that θ₄ is always the largest amongst them, so the definition of the cone angle may be reduced to:

$$ConeAngle = 2 \times min ( {θ}_{1} , {θ}_{2} , {θ}_{3} )$$

In accordance with certain embodiments of the present invention, the largest possible workspace is provided by configuring the dimensions and form of the spherical arms 16a, 16b, 16c such that α=β=γ. In such embodiments, the following is true:

$$\{ \begin{matrix}{θ}_{1} = - α + β + γ \\ {θ}_{2} = α - β + γ \\ {θ}_{3} = α + β - γ \\ {θ}_{1} = {θ}_{2} = {θ}_{3}\end{matrix}$$

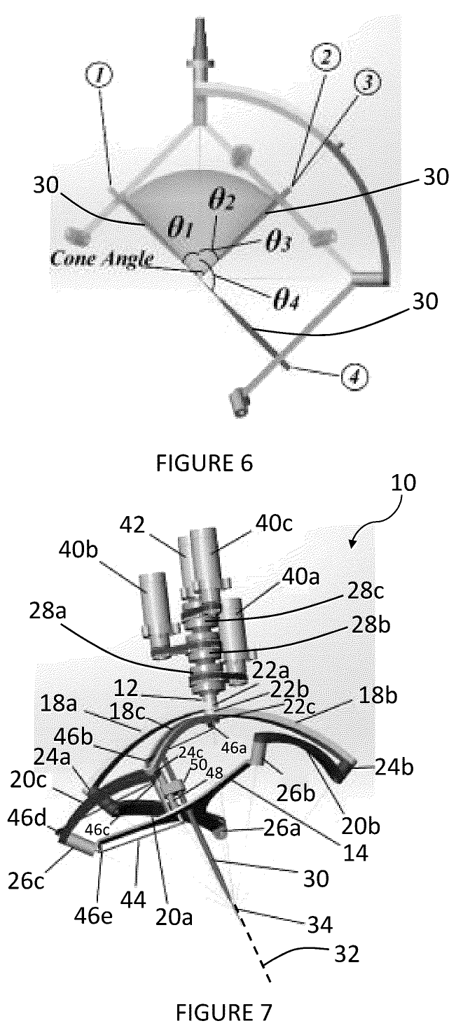

FIG. 6 shows an apparatus 10 according to an embodiment of the present invention with the maximum permitted cone angle (and hence the maximum permitted workspace) in which α=β=γ and θ₁=θ₂=θ₃. In accordance with other embodiments of the present invention, these relationships may not be defined as such, such that a non-maximum workspace may be provided.

In certain embodiments, a fourth degree of freedom may be provided permitting the tool 32 to be manipulated in a further manner in addition to the three degrees of freedom discussed above. In such embodiments, the fourth degree of freedom may be a translational movement of the tool 30 along the tool axis 32. However, the fourth degree of freedom is not limited to such. For example, in other embodiments, the fourth degree of freedom may be a clamping or grasping motion of the tool 30. In preferable embodiments, the fourth degree of freedom is provided without utilizing an actuator on the platform 14. In certain embodiments, examples of which are described in further detail below, an actuator that causes movement of the tool in accordance with the fourth degree of freedom is provided on the base 12. In certain embodiments, additional degrees of freedom beyond the fourth degree of freedom may also be provided. Such additional degrees of freedom may be provided without utilizing an actuator on the platform 14, and may optionally be provided by utilizing one or more actuators provided on the base 12. Any of the additional degrees of freedom beyond the three above-described degrees of freedom may comprise any suitable motion, including but not limited to one of translation of the tool 30 along the tool axis 32, clamping motion of the tool 30, and grabbing motion of the tool 30 (the latter two motions requiring the tool to have two or more articulatable parts that are moveable relative to one another).

As an illustrative example, FIG. 7 shows an apparatus 10 having four degrees of freedom in accordance with an embodiment of the present invention. The apparatus 10 is largely the same as the apparatus having three degrees of freedom described above with reference to FIGS. 1 to 6, with the exception that the apparatus of FIG. 7 is capable of translating the tool 30 along the tool axis 32 without reliance on any actuator on or around the platform 14. Instead, the apparatus 10 is provided with a cable 44 for causing movement of the tool in accordance with the fourth degree of freedom. The cable 44 extends from the base 12 to the platform 14 via a series of pulleys 46a, 46b, 46c, 46d, 46e. In particular, in the non-limiting embodiment shown in FIG. 7, the series of pulleys 46a, 46b, 46c, 46d, 46e are arranged so that the cable 44 extends from the base 12, along the first spherical linkage 18c, and the second spherical linkage 20c to the platform 14. In other embodiments, the cable 44 may extend along others of the first spherical linkages 18a, 18b and the second spherical linkages 20a, 20b. Upon reaching the (vicinity of the) platform 14, the cable 44 couples to a leadscrew 48. A nut 50 threaded on the leadscrew 48 holds the tool 30 relative to the platform 14. The cable 44 may be driven by an auxiliary actuator 42 mounted on the base 12 to cause rotation of the leadscrew 48. In the non-limiting embodiment shown in FIG. 7, the cable 44 is an “open loop” cable in that it terminates at its ends (as opposed to forming a continuous “closed loop”). Rotation of the leadscrew 48 causes the nut 50 and the tool 30 held by the nut 50 to translate in a direction parallel to the tool axis 32 (with the tool 30 translating directly along the tool axis 32) thereby providing the fourth degree of freedom. To facilitate the passage of the cable 44 from the base 12 to the platform 14, any one or more of the primary joints 22a, 22b, 22c, the first revolute joints 24a, 24b, 24c, and the second revolute joints 26a, 26b, 26c may be hollow joints and/or any one or more of the first spherical linkages 18a, 18b, 18c and the second spherical linkages 20a, 20b, 20c may have recesses, slots or other formations to accommodate the presence or passage of the cable 44. Alternatively, the cable 44 may extend from the base 12 to the platform 14 by one or more pulleys directly without passing along the first spherical linkages 18a, 18b, 18c and the second spherical linkages 20a, 20b, 20c.

As noted above, in other embodiments, the fourth degree of freedom may correspond to a movement or action of the tool 30 that is not translational along the tool axis 32. Such embodiments may still rely upon a cable being driven across a series of pulleys, wherein the cable is driven by an actuator mounted on the base 12. The platform 14 may comprise other components to translate the movement of the cable to a particular tool 30 action.

In certain embodiments, multiple pulleys may be provided to transfer drive from actuators mounted to the base 12 to the tool 30. In such embodiments, the three above-described degrees of movement will still be provided to permit movement of the tool 30 to various positions within the workspace and rotation of the tool 30 about the tool axis 32. In the non-limiting embodiment shown in FIG. 7, three primary actuators 40a, 40b, 40c are provided on the base 12 that drive the rotary inputs 28a, 28b, 28c via belts in order to provide the three degrees of freedom. In alternative embodiments, other actuators may be provided to provide drive to the rotary inputs 28a, 28b, 28c.

A further alternative embodiment of the present invention is shown in FIG. 8. The embodiment of FIG. 8 is similar to that of FIG. 7, however in place of the open loop cable 44 of FIG. 7, the embodiment of FIG. 8 has a closed loop cable 144 that is driven by an auxiliary actuator 42. The open loop cable 144 is guided from the base 12 to the (or near the) tool 30 along a series of pulleys 146a, 146b, 146c, 146d, 146e, 146f, 146g. In the non-limiting embodiment shown in FIG. 8, each of the series of pulleys 146a, 146b, 146c, 146d, 146e, 146f, 146g is, in effect, a double pulley in that it may permit the movement in opposing directions of both parts of the closed loop cable 144. In contrast to the embodiment of FIG. 7, the series of pulleys 146a, 146b, 146c, 146d, 146e, 146f, 146g in the embodiment of FIG. 8 guide the cable 144 along upper surfaces of the spherical arms 16a, 16b, 16c, thereby negating any need for any recesses, slots or other formations to accommodate the presence or passage of the cable 144. Similarly, the need for hollow revolute joints may be negated. Moreover, the cable 144 may be guided along the upper surface of a single one of the spherical arms 16a. The driven cable 144 rotates the leadscrew 48 and causes the nut 50 and the tool 30 held by the nut 50 to translate in a direction parallel to the tool axis 32 (with the tool 30 translating directly along the tool axis 32) thereby providing the fourth degree of freedom.

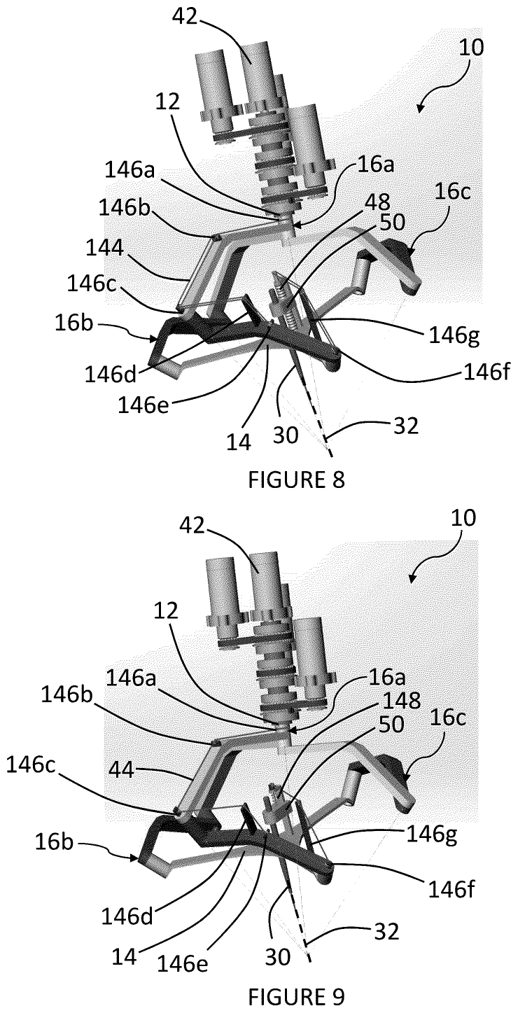

A further alternative embodiment of the present invention is shown in FIG. 9. The embodiment of FIG. 9 is identical to that of FIG. 8, however the closed loop cable 144 is replaced with an open loop cable 44 (as described above with reference to FIG. 7) and the leadscrew is replaced with an arrangement in which the cable 44 is directly connected to the nut 50, with biasing means in the form of an extension spring 148 being provided (in other embodiments, other biasing means may be provided). The driven cable 44 may retract the nut 50 and the tool 30 connected thereto from a first position to a second position along the tool axis 32. In doing so, the extension spring 148 is compressed and may act to return to nut 50 and tool 30 to the first position (by acting between the nut 50 and another reaction surface). Consequently, the tool 30 may be moved back and forth (between the first and second positions) along the tool axis 32 by the cable 44 thereby providing the fourth degree of freedom.

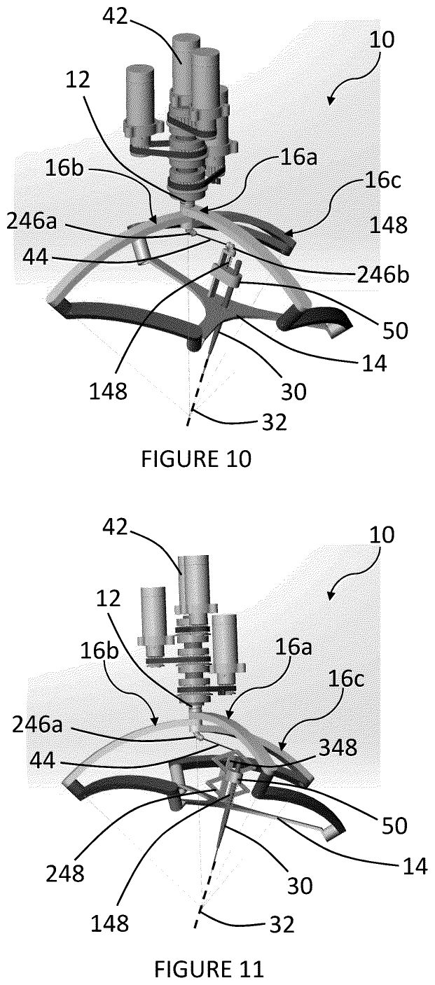

A further alternative embodiment of the present invention is shown in FIG. 10. The embodiment of FIG. 10 is identical to that of FIG. 9, however the cable 44 is guided directly from the base 12 to the nut 50 without passing over a series of pulleys provided on any of the spherical arms 16a, 16b, 16c. Instead, the cable 44 passes directly from the base 12 to the nut 50. A pair of cable guides, 246a, 246b is provided. In particular, a first cable guide 246a is proximate to the base 12, and a second cable guide 246b is proximate to the nut 50 to guide the cable 44. The cable 44 is driven as it is in the embodiment of FIG. 9 in order to translate the tool 30 along the tool axis 32, thereby providing the fourth degree of freedom.

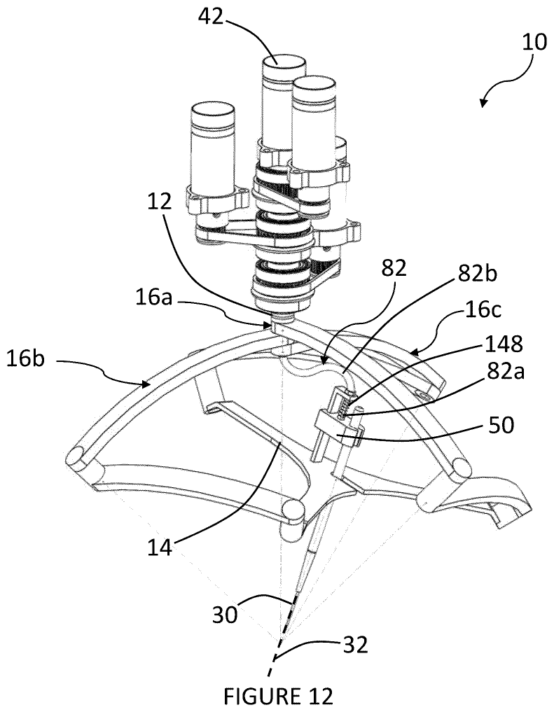

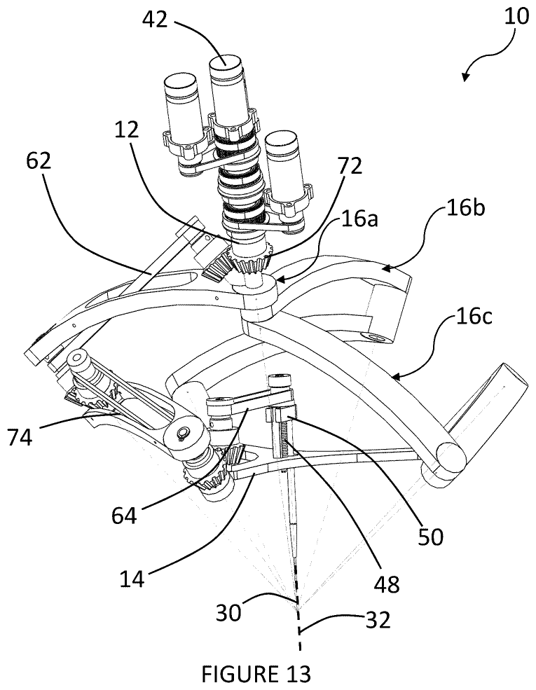

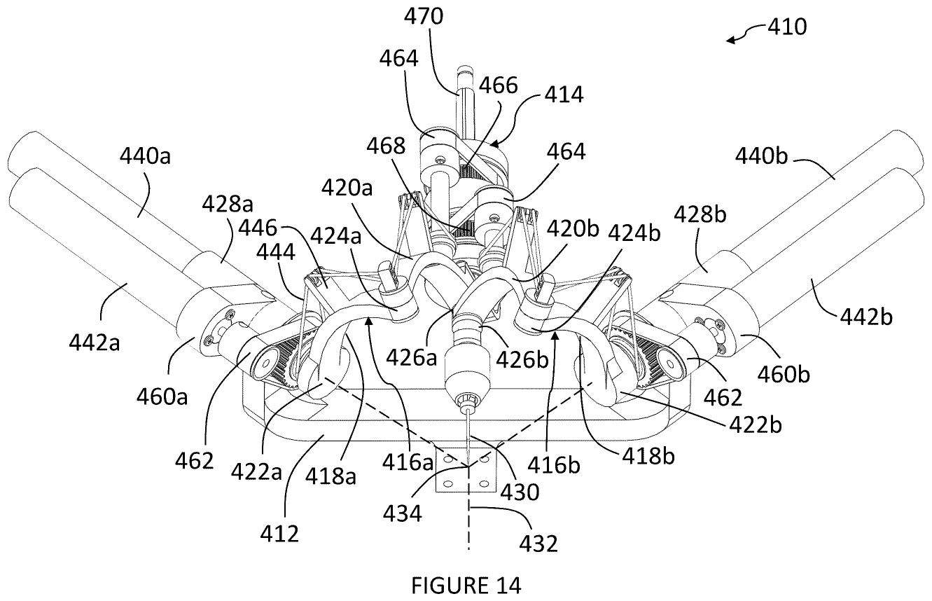

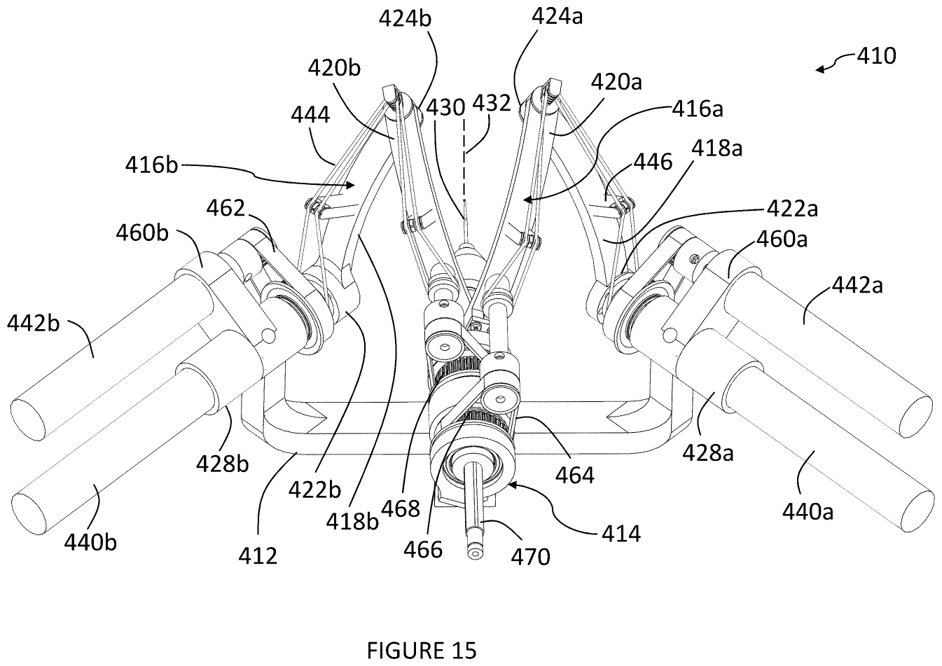

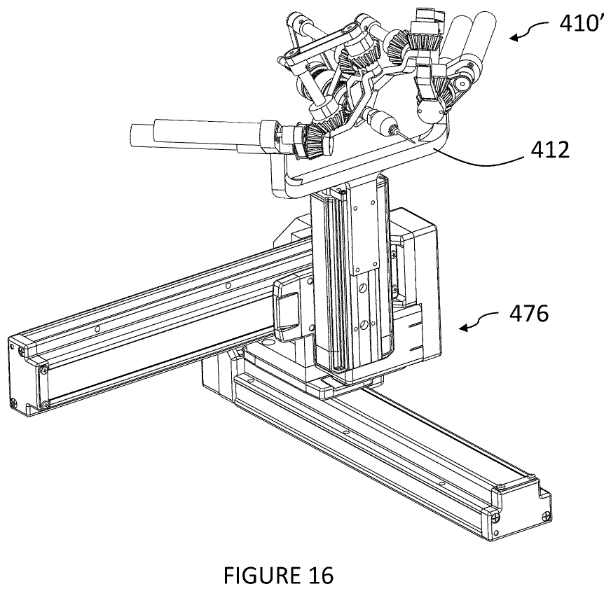

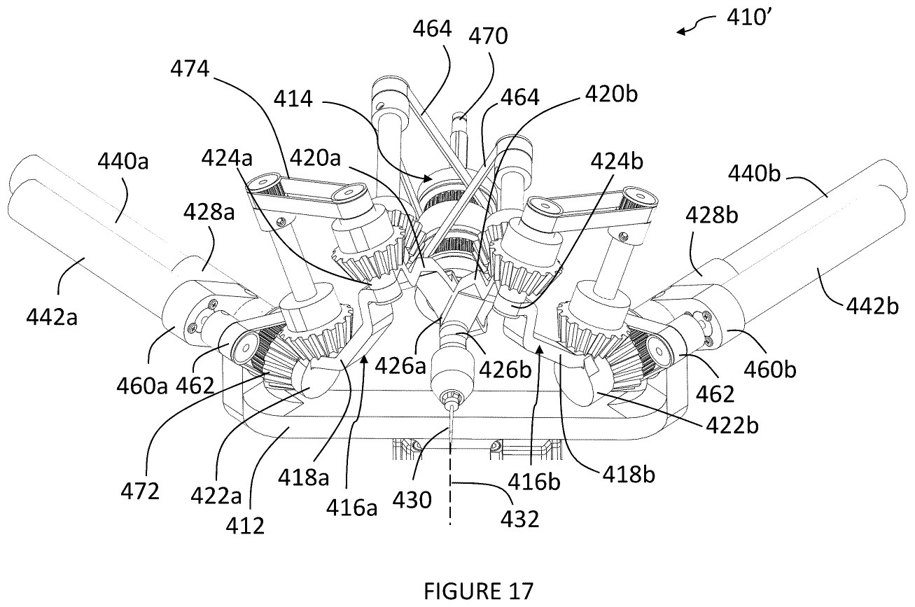

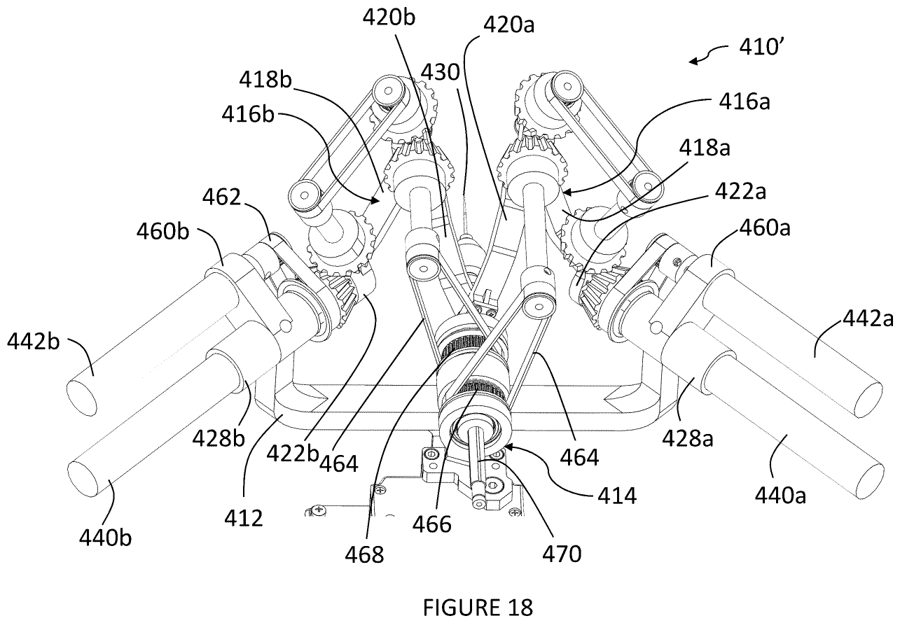

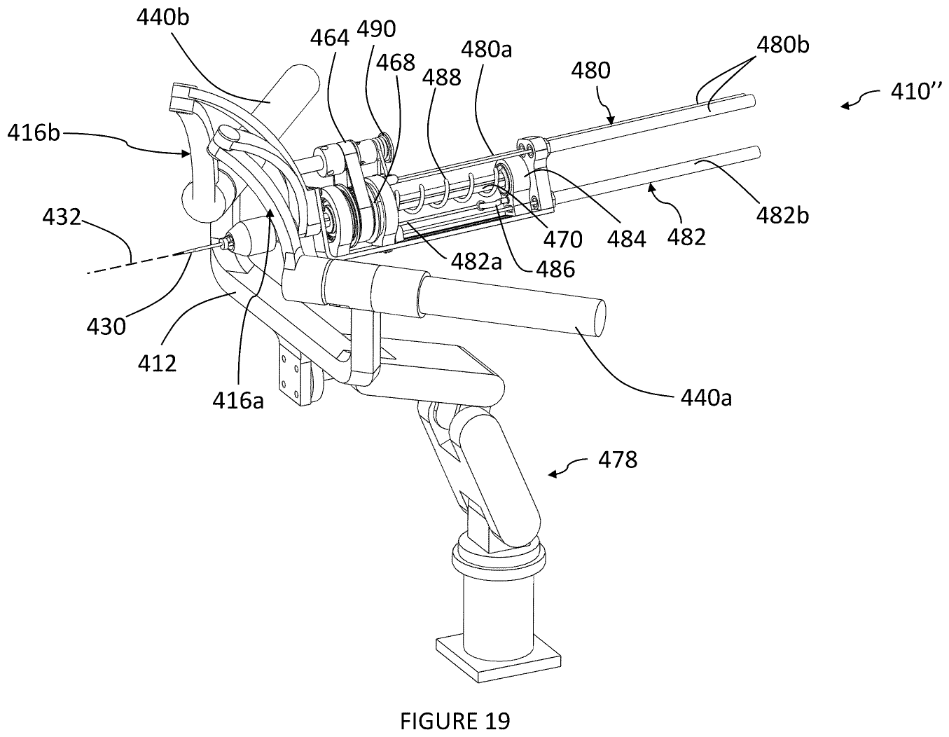

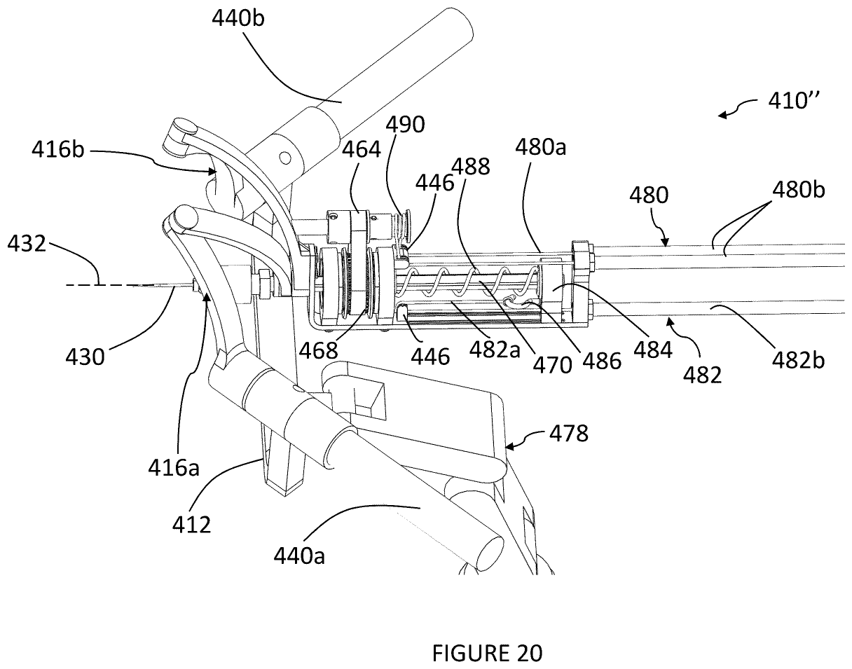

A further alternative embodiment of the present invention is shown in FIG. 11. The embodiment of FIG. 11 is identical to that of FIG. 10, however the second cable 246b is replaced by a scissor mechanism 248 that may be extended relative a frame 348 on the platform 14 by retraction of the cable 44 from a first position to a second position. An extension spring 148 is provided and is arranged to extend as the scissor mechanism 248 moves from the first position to the second position. In extending, the extension spring 148 acts to bias the scissor mechanism back to the first position. The nut 50 is affixed relative to the scissor mechanism 248 and the tool 30 is affixed to the nut 50 such that movement of the scissor mechanism 248 causes movement of the tool 30 along the tool axis 32, thereby providing the fourth degree of freedom. In alternative embodiments, the second cable guide may additionally be provided. In certain embodiments, other extension means may be provided in place of the scissor mechanism and/or other biasing means may be provided in place of the extension spring 148.

In certain embodiments, the whole apparatus 10 may itself be moveable so as to move the tool 30 beyond the range of movement permitted by the apparatus 10. For example, the apparatus 10 may be mounted to an articulatable arm.

By virtue of the nature of the motion permitted by apparatuses according to embodiments of the present invention, such apparatuses may be termed coaxial spherical parallel mechanisms.

As noted above, certain embodiments of the present invention are particularly suited for use in minimally invasive surgical procedures. In particular, embodiments of the present invention are particularly suited to positioning and manipulating a surgical tool during a surgical procedure. Examples of applicable minimally invasive surgical procedures include, but are not limited to, laparoscopic surgery, heart surgery, endovascular surgery, cancer surgery, and eye surgery. Use of embodiments of the present invention may overcome or mitigate certain disadvantages associated with prior art surgical methods that suffer from limited vision of the surgical site, hand tremor, existing leverage effect, lack of force feedback, restricted degree of freedom, and the risk of damaging surrounding tissues.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. An apparatus for positioning a tool, the apparatus comprising:
a base;
a platform for supporting the tool;
three spherical arms each connecting the platform to the base, each spherical arm comprising a first spherical linkage and a second spherical linkage, wherein:
(i) each first spherical linkage is connected to the base by a primary joint, and all primary joints are coaxial with one another;
(ii) each first spherical linkage is connected to the respective second spherical linkage by a first revolute joint;
(iii) each second spherical linkage is connected to the platform by a second revolute joint; and
(iv) axes of the primary joints, first revolute joints and second revolute joints intersect at a remote center of motion that is remote from the platform; and
rotary inputs arranged on the base to receive rotary drive to rotate each first spherical linkage about the respective primary joint so as to move the platform over a portion of the surface of a notional sphere and rotate the platform about a tool axis that is normal to the surface of the platform;
the apparatus further comprising one or more cables extending from the base to the platform, wherein the one or more cables is each drivable to move the tool supported on the platform to provide a further degree of freedom of the tool.

2. An apparatus according to claim 1, wherein the three spherical arms are configured such that all extreme permitted positions of the tool axis relative to the axes of the primary joints are outside of a conical workspace that is centered on and symmetrical about the axes of the primary joints.

3. An apparatus according to claim 2, wherein for four extreme positions, 1, 2, 3, 4 of the tool axis, a cone angle of the workspace is equal to twice the minimum angle amongst θ₁, θ₂, θ₃, and θ₄, wherein:
$$\{ \begin{matrix}{θ}_{1} = - α + β + γ \\ {θ}_{2} = α - β + γ \\ {θ}_{3} = α + β - γ \\ {θ}_{4} = α + β + γ\end{matrix} ;$$
and
α is the angle between the primary joints and a respective first revolute joint, β is the angle between the first revolute joint and a respective second revolute joint, and γ is the angle between the second revolute joint and the tool axis.

4. An apparatus according to claim 3, wherein the three spherical arms are configured such that α=β=γ and θ₁=θ₂=θ₃.

5. An apparatus according to claim 1, comprising a tool supported by the platform.

6. An apparatus according to claim 5, wherein the tool is a surgical tool.

7. (canceled)

8. An apparatus according to claim 1, wherein the one or more cables extend from the base to the platform over one or more pulleys mounted to none, one or more than one of the three spherical arms.

9. An apparatus according to claim 1, wherein the further degree of freedom of the tool comprises one or more of translation of the tool along the tool axis, a grabbing motion of the tool or a clamping motion of the tool.

10. An apparatus according to claim 9, wherein the further degree of freedom comprises translation of the tool along the tool axis, and the apparatus further comprises a leadscrew and a nut threaded on the leadscrew, wherein the nut supports the tool relative to the platform and is translatable along the leadscrew so as to move the tool along the tool axis in response to the leadscrew being driven by one of the one or more cables.

11. An apparatus according to claim 1, wherein one or more of the three spherical arms comprise recesses to accommodate the one or more cables.

12. An apparatus according to claim 1, wherein one or more of the primary joints, the first revolute joints and the second revolute joints are hollow so as to permit the passage of the one or more cables.

13. An apparatus according to claim 1, comprising one or more auxiliary actuators arranged on the base and each configured to drive the one of the one or more cables.

14. An apparatus according to claim 1, comprising one or more primary actuators arranged on the base and each configured to provide rotary drive to one of the rotary inputs.

15. An apparatus for positioning a tool, the apparatus comprising:
a base;
a platform for supporting the tool;
three spherical arms each connecting the platform to the base, each spherical arm comprising a first spherical linkage and a second spherical linkage, wherein:
(i) each first spherical linkage is connected to the base by a primary joint, and all primary joints are coaxial with one another;
(ii) each first spherical linkage is connected to the respective second spherical linkage by a first revolute joint;
(iii) each second spherical linkage is connected to the platform by a second revolute joint; and
(iv) axes of the primary joints, first revolute joints and second revolute joints intersect at a remote center of motion that is remote from the platform; and
rotary inputs arranged on the base to receive rotary drive to rotate each first spherical linkage about the respective primary joint so as to move the platform over a portion of the surface of a notional sphere and rotate the platform about a tool axis that is normal to the surface of the platform;
the apparatus further comprising a plurality of bevel gears and a plurality of belts each drivably coupled to at least one of the bevel gears, the plurality of bevel gears and belts extending from the base to the platform, wherein the plurality of bevel gears and belts are drivable to move the tool supported on the platform to provide further degree of freedom of the tool.

16. An apparatus according to claim 15, wherein the bevel gears and belts are mounted to none, one or more than one of the three spherical arms.

17. An apparatus for positioning a tool, the apparatus comprising:
a base;
a platform for supporting the tool;
three spherical arms each connecting the platform to the base, each spherical arm comprising a first spherical linkage and a second spherical linkage, wherein:
(i) each first spherical linkage is connected to the base by a primary joint, and all primary joints are coaxial with one another;
(ii) each first spherical linkage is connected to the respective second spherical linkage by a first revolute joint;
(iii) each second spherical linkage is connected to the platform by a second revolute joint; and
(iv) axes of the primary joints, first revolute joints and second revolute joints intersect at a remote centre of motion that is remote from the platform; and
rotary inputs arranged on the base to receive rotary drive to rotate each first spherical linkage about the respective primary joint so as to move the platform over a portion of the surface of a notional sphere and rotate the platform about a tool axis that is normal to the surface of the platform;
the apparatus further comprising one or more Bowden cables extending from the base to the platform wherein each of the one or more Bowden cables is drivable to move the tool supported on the platform to provide a further degree of freedom of the tool.

18. An apparatus according to claim 17, wherein the further degree of freedom comprises translation of the tool along the tool axis, and the apparatus further comprises a nut arranged to support the tool relative to the platform, the nut being connected to and moveable by the one or more Bowden cables so as to move the tool along the tool axis.

19-36. (canceled)

37. A system comprising an apparatus according to claim 1, and an XYZ Cartesian stage or an articulatable arm supporting the apparatus.

38. (canceled)

39. A system comprising an apparatus according to claim 15, and an XYZ Cartesian stage or an articulatable arm supporting the apparatus.

40. A system comprising an apparatus according to claim 17, and an XYZ Cartesian stage or an articulatable arm supporting the apparatus.
